(19) United States
(12) Patent Application Publication
(10) Pub. No.: US 2026/0256417 A1
(43) Pub. Date: Sep. 3, 2026

(54) METHOD AND SYSTEM FOR FACIAL SKIN COMPONENT IMAGE SEPARATION

(71) Applicant: SHENZHEN HYPERNANO OPTICS TECHNOLOGY CO., LTD., Shenzhen (CN)

(72) Inventors: Bin GUO, Shenzhen (CN); Xingchao Yu, Shenzhen (CN); Zhe Ren, Shenzhen (CN); Jinbiao Huang, Shenzhen (CN)

(21) Appl. No.: 18/717,060

(22) PCT Filed: Jan. 30, 2023
(86) PCT No.: PCT/CN2023/073829

§ 371 Date: Jun. 6, 2024

(30) Foreign Application Priority Data
Dec. 6, 2021 (CN) 202111477430.9

(51) Int. Cl. A61B 5/00; A61B 5/145; A61B 5/1455; G06T 5/40; G06T 5/50; G06T 5/70; G06T 5/92; G06T 7/00; G06T 7/90

(52) U.S. Cl. CPC A61B 5/443; A61B 5/14546; A61B 5/1455; G06T 5/40; G06T 5/50; G06T 5/70; G06T 5/92; G06T 7/0016; G06T 7/90; A61B 2576/02; G06T 2207/10024; G06T 2207/20224; G06T 2207/30088; G06T 2207/30101; G06T 2207/30201

(57) ABSTRACT

Disclosed in the present application are a separation method and system for image with facial skin component, comprising: collecting facial hyperspectral image data in different preset wavebands, which wavebands have obvious relative changes of hemoglobin component and melanin; performing white balance processing or absolute reflectance processing on the face hyperspectral image data in the different wavebands; using an image processing algorithm to obtain an original hemoglobin component distribution image, constructing a skin reflection model, and obtaining content distributions of melanin and hemoglobin component by making linear regression on pixels comprising concentrations of melanin and hemoglobin component; using an image processing algorithm to obtain a melanin content difference distribution image, and obtaining a melanin distribution image based on the content distribution of melanin component, wherein the image processing algorithm comprises image subtraction or image division. The present invention uses simple calculation to separate the two major components of the face skin, namely hemoglobin and melanin, from a hyperspectral image captured with the fewest number of wavebands.

### CROSS REFERENCE TO RELATED APPLICATION(S)

This application claims priority of a Chinese patent application CN202111477430.9 filed on Dec. 6, 2021, the entirety of which is incorporated by reference.

### TECHNICAL FIELD

The application relates to the technical field of hyperspectral analysis, in particular to a separation method and system for image with facial skin component.

### BACKGROUND

Hyperspectral imaging technology can obtain image information and spectral information at the same time by collecting images of the same scene at different wavelengths. When combining with machine vision and other technologies to identify objects, it can also perform spectral analysis based on spectral characteristics. The spectral wave bandwidth (Full Width at Half Maximum (FWHM)) of three channels of ordinary RGB color imaging is about 80 nm-100 nm, while the hyperspectral imaging corresponding to a wavelength position can usually collect narrow waveband information with a FWHM of about 2 nm-20 nm on the spectrum. The spectral analysis ability of hyperspectral imaging technology comes from the spectral information of substances collected by hyperspectral in a certain spectral range (usually corresponding to a spectral range of 100-400 nm), which directly reflects various useful physical and chemical compositions of objects. Combined with image recognition, region selection and other information, hyperspectral imaging technology can realize the complete automation of object detection, component judgment and result output. Hyperspectral image analysis can be applied to a wide range of fields, comprising medical field and cosmetic field.

Medical and cosmetic professionals usually need to detect the skin condition comprising the face. In most cases, the characteristics needed to be detected are the changes of skin color. However, the detection of these symptoms currently faces several challenges, one of which is that some changes are not clearly visible to eyes and are easily overlooked, and the other is that some symptoms cannot be accurately distinguished from each other. Skin color is mainly determined by the pigments in the skin, the main pigments in the skin are melanin and hemoglobin. High contents of melanin and hemoglobin are also markers of various skin diseases. Melanin is distributed at different depths of the skin and is the main component of skin color. In normal healthy skin, melanin particles are small and evenly distributed, resulting in a smooth surface and uniform skin color. An increased melanin deposition is usually caused by prolonged exposure to sunlight or skin diseases, such as acne. Therefore, the deposition of melanin will negatively affect the uniformity of skin color. Hemoglobin is present in the papillary layer of dermis in both Oxy and Deoxy forms and constitutes the red color of the skin. Some skin conditions, such as acne, rosacea and telangiectasia, can cause organic changes in the vascular structure of patients and increase the level of hemoglobin in the dermis. The increase in the number of hemoglobin and the formation of new vascular structures will lead to the red color of the skin and have a negative impact on the uniformity of skin color.

As early as the 1920s, people began using various methods to measure skin pigmentation. At that time, people believed that “the more melanin deposited, the more light absorbed, the darker the skin color”. But skin color is not only determined by melanin. Hemoglobin can also absorb visible light. So a method is needed to clearly distinguish between melanin and hemoglobin in the skin. There are currently two main methods for distinguishing the distribution of melanin and hemoglobin in the skin: the first is the VISIA imaging system based on RBX technology, which uses polarized light as the light source and uses an RGB camera as the capture device, the melanin and hemoglobin on the skin are separated by RBX technology. However, this method has a cumbersome device, the separated melanin and hemoglobin are not well interpretable, and some areas cannot be separated completely. The other method is skin spectroscopy technology, which is a multi-spectral technology that uses different colored polarized light sources to illuminate the skin and uses an RGB camera to capture images. The disadvantage of this method is that it requires different colored polarized light sources and takes longer to capture images.

### SUMMARY

The above skin pigmentation analysis methods have problems such as complex data analysis processes for extracting spectral information of substances, complex structures of shooting equipment, and low analysis accuracy. The embodiment of this application provides a separation method for image with facial skin component to solve the problems above.

According to the first aspect of the application, a separation method for image with facial skin component, comprising the following steps of:
S1: collecting facial hyperspectral image data in different preset wavebands, which wavebands have obvious relative changes of hemoglobin component and melanin;
S2: performing white balance processing or absolute reflectance processing on the face hyperspectral image data in the different wavebands;
S3: using an image processing algorithm to obtain an original hemoglobin component distribution image, constructing a skin reflection model, and obtaining content distributions of melanin and hemoglobin component by making linear regression on pixels comprising concentrations of melanin and hemoglobin component; and
S4: using an image processing algorithm to obtain a melanin content difference distribution image, and obtaining a melanin distribution image based on the content distribution of melanin component, wherein the image processing algorithm comprises image subtraction or image division.

In some specific embodiments, the facial hyperspectral image data is taken by a hyperspectral imaging camera with a Full Width at Half Maximum (FWHM) less than 50 nm. The hyperspectral imaging camera with a small FWHM can ensure the clear effect of separating face components, so as to a stricter physical significance.

In some specific embodiments, the facial hyperspectral image data of three different preset wavebands is collected, and the white balance processing in step S2 comprises: obtaining the white balance by using the Gray World Algorithm, and obtaining three frames of facial hyperspectral images I₁(x, y), I₂(x, y), and I₃(x, y) after the white balance.

In some specific embodiments, the facial hyperspectral image data of three preset different wavebands is collected, and the absolute reflectance processing in step S2 comprises: obtaining hyperspectral data of at original facial position of a reference white board in corresponding waveband, dividing the facial hyperspectral images by the hyperspectral data of the reference white board, and obtaining three frames of facial hyperspectral images I₁(x, y), I₂(x, y), and I₃(x, y) after the absolute reflectance processing.

The two methods above can expand the application scenario and greatly increase the flexibility and portability of the application.

In some specific embodiments, the three preset different wavebands comprise 530-560 nm, 575-585 nm and 600-630 nm, and the three frames of facial hyperspectral images in the corresponding three wavebands are obtained. It can be approximated that the contents of melanin in wavebands near 580 nm and 620 nm is unchanged, and then the content and distribution of hemoglobin can be calculated by the difference. The absorption rate of hemoglobin at the bimodal feature of 530 nm to 585 nm is nearly unchanged and almost equal, while melanin decreases exponentially, therefore it can choose two bimodal wavebands of 530 nm to 550 nm and 580 nm to 585 nm to eliminate hemoglobin and obtain melanin components.

In some specific embodiments, the original hemoglobin component distribution image is obtained by using the image processing algorithm, comprising subtracting the facial hyperspectral image I₂(x, y) by I₃(x, y), or dividing I₂(x, y) by I₃(x, y) to obtain the original hemoglobin component distribution image O(x, y).

In some specific embodiments, the constructing a skin reflection model comprises: based on the Lambert-Beer law, linear regression is performed on each of the pixels by combining light absorbance values of hemoglobin and melanin in two wavebands and the hyperspectral image data captured: −Log(R)=CoO+CMM, image information in two wavebands is used to obtain linear equation in two unknowns of Co and CM, and the concentration or content of hemoglobin and melanin at each pixel position is obtained, where R represents the reflectance of facial skin, O and M represent the light absorbance coefficients of hemoglobin and melanin respectively, Co and CM represent the corresponding concentration or content of hemoglobin and melanin. With this step, the separation of the two major skin components can be accomplished without extensive calculation.

In some specific embodiments, obtaining the melanin content difference distribution image in the step S4 comprises: obtaining the melanin content difference distribution image ΔM(x, y) by subtracting the facial hyperspectral image I₁(x, y) by the I₂(x, y) which is processed with Gaussian blur, or by dividing the I₁(x, y) by the I₂(x, y) which is processed with Gaussian blur.

In some specific embodiments, obtaining the melanin distribution image comprises: obtaining the melanin distribution image M(x, y) based on the image processing algorithm, M(x, y)=I₃(x, y)+ΔM(x, y), or obtaining the melanin distribution image M(x, y) based on the skin reflection model, M(x, y)=M′(x, y)+ΔM(x, y), where M′(x, y) is the content distribution of melanin component obtained by the linear regression.

In some specific embodiments, the separation method further comprises image enhancement processing on gray-scale images of contents of hemoglobin and melanin, and the image enhancement processing comprises maximum-minimum normalization, contrast enhancement, and histogram equalization.

According to the second aspect of the application, a separation system for face skin components is proposed, which comprises:
Hyperspectral image collecting unit: configured for collecting facial hyperspectral image data in different preset wavebands, which wavebands have obvious relative changes of hemoglobin component and melanin;
Hyperspectral image processing unit: configured for performing white balance processing or absolute reflectance processing on the face hyperspectral image data in the different wavebands;
Hemoglobin component distribution image obtaining unit: configured for using an image processing algorithm to obtain an original hemoglobin component distribution image, constructing a skin reflection model, and obtaining content distributions of melanin and hemoglobin component by making linear regression on pixels comprising concentrations of melanin and hemoglobin component; and
Melanin distribution image obtaining unit: configured for using an image processing algorithm to obtain a melanin content difference distribution image, and obtaining a melanin distribution image based on the content distribution of melanin component, wherein the image processing algorithm comprises image subtraction or image division.

In some specific embodiments, it further comprises an image enhancement processing unit configured for performing image enhancement processing on gray-scale images of contents of hemoglobin and melanin, and the image enhancement processing comprises maximum-minimum normalization, contrast enhancement, and histogram equalization.

According to the third aspect of the application, a computer readable storage medium storing one or more computer programs is proposed, the computer programs implement any one of the methods above when executed by a computer processor.

The application provides a separation method and system for image with facial skin component, which can stably and reliably display the two principal components of face skin, hemoglobin and melanin, from hyperspectral images captured with the fewest number of spectral wavebands (three), and have a very good visual display effect. And it is still effective in camera obscura, or the open environment with strictly control of lighting, the images captured by this method do not require taking a whiteboard or color card for color correction or white balance correction, which can expand the application scenario and greatly increase the flexibility and portability of the application. In addition, compared to ordinary RGB cameras with wide wavebands, the separated hemoglobin and melanin components are clearer and have stricter physical meanings due to the use of narrow waveband,

### BRIEF DESCRIPTION OF THE DRAWINGS

Comprising accompanying drawings to provide a further understanding of the embodiments and the drawings are incorporated into this specification and form part of this specification. The drawings illustrate the embodiments and are used in conjunction with the description to explain the principles of the present disclosure. Many expected advantages of other embodiments and embodiments will be easily understood referring to the following detailed description. The elements of the drawings are not necessarily proportional to each other. The same drawing symbols refer to corresponding similar parts.

FIG. 1 is a flow chart of the separation method for image with facial skin component in one embodiment of the application;

FIG. 2 is a spectrum of absorption rates of hemoglobin and melanin in the visible light range in a specific embodiment of the application;

FIG. 3 is an effect diagram of image enhancement of a gray-scale image of desired the contents of hemoglobin and melanin in a specific embodiment of the application;

FIG. 4 is a flow chart of the separation method for image with facial skin component in a specific embodiment of the application;

FIG. 5 is an effect diagram of separation at different single-waveband FWHM in the embodiment of the application;

FIG. 6 is intensity difference curves of hemoglobin and melanin at different single-waveband FWHM in the embodiment of the application;

FIG. 7 is a block diagram of the separation system for image with facial skin component in one embodiment of the application;

FIG. 8 is a schematic diagram of the structure of a computer system suitable for implementing the embodiment of the present application.

### DETAILED DESCRIPTION OF THE DISCLOSURE

This application is to be further described in detail below in combination with the accompanying drawings and embodiments. It is understood that the specific embodiments described herein are only used to explain the relevant application, not to define the application. It should further be noted that only the parts of the application are shown for ease of description.

It should be noted that, the embodiments and features in the embodiments of this application can be combined with each other without conflict. This application is described in detail below in combination with the accompanying drawings and embodiments.

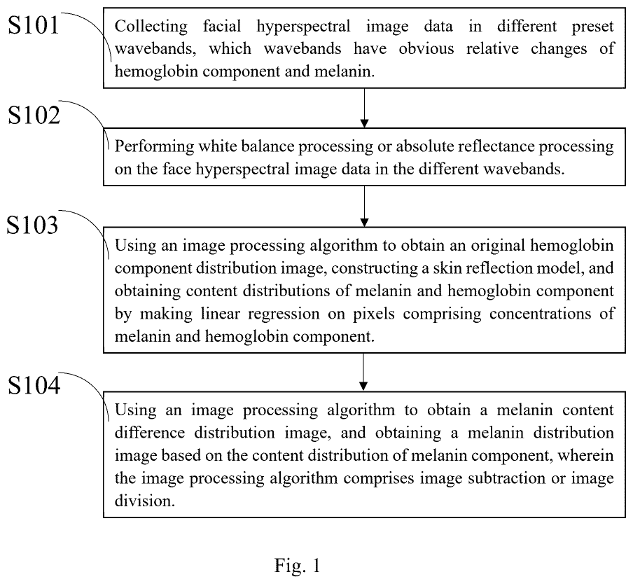

As shown in FIG. 1, the embodiment of the present application provides a separation method for image with facial skin component, comprising the following steps:

S101: Collecting facial hyperspectral image data in different preset wavebands, which wavebands have obvious relative changes of hemoglobin component and melanin.

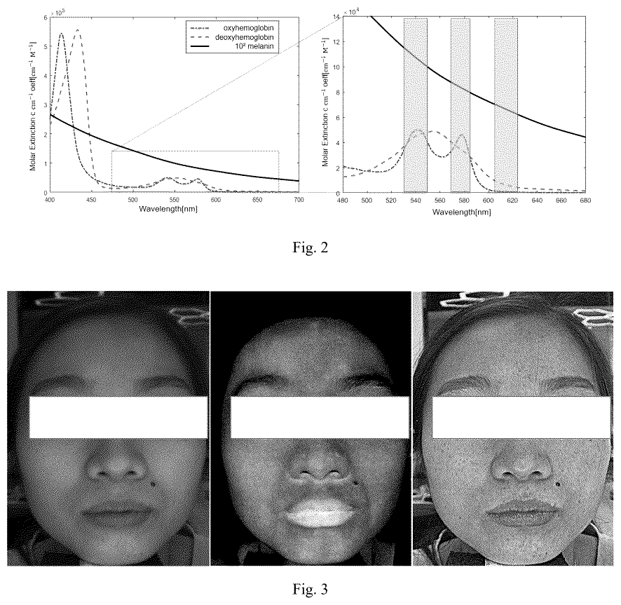

In specific embodiments, the three preset different wavebands comprise 530-560 nm, 575-585 nm and 600-630 nm, and the three frames of facial hyperspectral images in the corresponding three wavebands are obtained. As the absorption rates of hemoglobin and melanin in shown in FIG. 2, the hemoglobin showed a sharp decline between 580 nm and 620 nm, while the melanin remained flat, so it can be approximated that the content of melanin in wavebands near 580 nm and 620 nm is unchanged, and then the content and distribution of hemoglobin can be calculated by the difference. The absorption rate of hemoglobin at the bimodal feature of 530 nm to 585 nm is nearly unchanged and almost equal, while melanin decreases exponentially, so it can choose two bimodal wavebands of 530 nm to 550 nm and 580 nm to 585 nm to eliminate hemoglobin and obtain melanin component. Therefore, the optimum wavelength for separating hemoglobin and melanin in practical applications can be obtained in three wavebands, which are respectively between 530~560 nm, 575~585 nm, and 600~630 nm. And it should also be recognized that there also exists different waveband selections to approach the technical effect of the invention.

S102: Performing white balance processing or absolute reflectance processing on the face hyperspectral image data in the different wavebands.

In specific embodiments, in one way, it can obtain three frames of facial hyperspectral images I₁(x, y), I₂(x, y), and I₃(x, y) by using the Gray World Algorithm to achieve the white balance. In another way, it can obtain hyperspectral data of at original facial position of a reference white board in the corresponding waveband, divide the facial hyperspectral images by the hyperspectral data of the reference white board, and then obtain three frames of facial hyperspectral images I₁(x, y), I₂(x, y), and I₃(x, y) after the absolute reflectance processing. It can expand the application scenario and greatly increase the flexibility and portability of the application for being able to works in camera obscura, or the open environment with strictly control of lighting.

S103: Using an image processing algorithm to obtain an original hemoglobin component distribution image, constructing a skin reflection model, and obtaining content distributions of melanin and hemoglobin component by making linear regression on pixels comprising concentrations of melanin and hemoglobin component.

In specific embodiments, the application obtains the original hemoglobin component distribution image O(x, y) by subtracting the facial hyperspectral image I₂(x, y) by I₃(x, y), or dividing I₂(x, y) by I₃(x, y). The constructing a skin reflection model comprises: based on the Lambert-Beer law, linear regression is performed on each of the pixels by combining light absorbance values of hemoglobin and melanin in two wavebands and the hyperspectral image data captured: −Log(R)=CoO+CMM. And image information in two wavebands is used to obtain linear equation in two unknowns of Co and CM, and the concentration or content of hemoglobin and melanin at each pixel position is obtained, where R represents the reflectance of facial skin, O and M represent the light absorbance coefficients of hemoglobin and melanin respectively, Co and CM represent the corresponding concentration or content of hemoglobin and melanin.

S104: Using an image processing algorithm to obtain a melanin content difference distribution image, and obtaining a melanin distribution image based on the content distribution of melanin component, wherein the image processing algorithm comprises image subtraction or image division.

In specific embodiments, obtaining the melanin content difference distribution image ΔM(x, y) by subtracting the facial hyperspectral image I₁(x, y) by the I₂(x, y) which is processed with Gaussian blur, or by dividing the I₁(x, y) by the I₂(x, y) which is processed with Gaussian blur. And obtaining the melanin distribution image M(x, y) based on the image processing algorithm, M(x, y)=I₃(x, y)+ΔM(x, y), or obtaining the melanin distribution image M(x, y) based on the skin reflection model, M(x, y)=M′(x, y)+ΔM(x, y), where M′(x, y) is the content distribution of melanin component obtained by the linear regression.

In specific embodiments, the separation method further comprises image enhancement processing on gray-scale images of contents of hemoglobin and melanin, so as to improve image contrast and visual presentation. The image enhancement processing comprises maximum-minimum normalization, contrast enhancement, and histogram equalization. Finally, the content images of hemoglobin and melanin components are obtained with obvious visual contrast. As shown in FIG. 3, the images from the left to the right are gray-scale image, hemoglobin distribution image and melanin distribution image respectively. In this embodiment, the Gray World Algorithm is adopted to achieve the white balance and the skin reflection model is used to separate hemoglobin and melanin. The final results obtained by applying the method in this application are all consistent with the distribution characteristics of hemoglobin and melanin: There is no hemoglobin content in the hair (comprising hair, eyelashes, eyebrows, etc.), and the highest content of hemoglobin is in the lips. The distributions of melanin are concentrated in hair, blackheads, and moles; In addition, hemoglobin is distributed in flaky areas, while melanin is distributed in dotted areas. The characteristics are consistent with the actual situation.

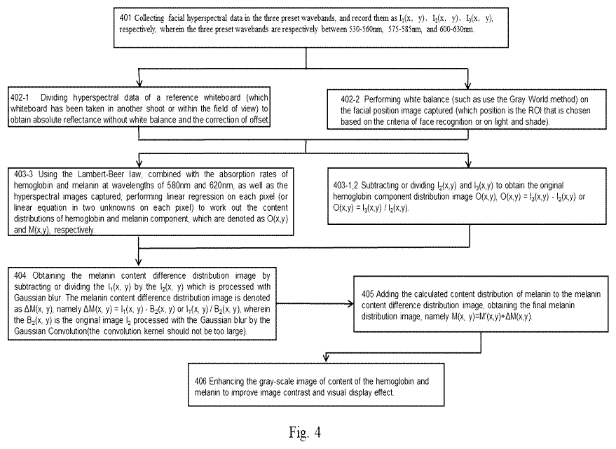

FIG. 4 illustrates a flow chart of the separation method for image with facial skin component in a specific embodiment of the application, as shown in FIG. 4, it comprises the following steps of:
S401: Collecting facial hyperspectral data in the three preset wavebands, and recording them as I₁(x, y), I₂(x, y), I₃(x, y), respectively, wherein the three preset wavebands are respectively between 530-560 nm, 575-585 nm, and 600-630 nm.
S402-1: Dividing hyperspectral data of a reference whiteboard (which whiteboard has been taken in another shoot or within the field of view) to obtain absolute reflectance without white balance and the correction of offset. Placing the reference whiteboard at the position of the face, photographing the hyperspectral data at the three corresponding wavebands, and recording them as W₁(x, y), W₂(x, y), and W₃(x, y), respectively. Then, dividing I₁(x, y), I₂(x, y), and I₃(x, y) by W₁(x, y), W₂(x, y), and W₃(x, y) correspondingly, to obtain the absolute reflectance of facial images in the three wavebands.
S402-2: Performing white balance (such as use the Gray World method) on the facial position image captured (which position is the ROI that is chosen based on the criteria of face recognition or on light and shade). Without photographing the reference whiteboard, performing image white balance directly on the face acquired in step 1. For example, the Gray World methods can be used to obtain white balance for facial position (For instance, the ROI selection of the face can be chosen by face recognition), then the three frames of the facial images after white balance can be updated to replace the original I₁(x, y), I₂(x, y), and I₃(x, y).
S403-1, 2: Subtracting or dividing I₂(x, y) by I₃(x, y) to obtain the original hemoglobin component distribution image O(x, y), O(x, y)=I₃(x, y)−I₂(x, y) or O(x, y)=I₃(x, y)/I₂(x, y). It should be noted that the two methods above can be used in practical applications in combination.
S403-3: Using the Lambert-Beer law, combined with the absorption rates of hemoglobin and melanin at wavelengths of 580 nm and 620 nm, as well as the hyperspectral images captured, performing linear regression on each pixel (or linear equation in two unknowns on each pixel) to work out the content distributions of hemoglobin and melanin component, which are denoted as O(x, y) and M(x, y), respectively.

In specific embodiments, constructing a simple skin reflection model (e.g. assuming it conforms to Lambert-Beer law). The actual skin reflection law is more complex than this, but the connection between the components can be approximately viewed as linearly independent and having a single reflective layer, thereby a simple mathematical model is established. Assuming the skin of each pixel is a combination of two components of melanin and hemoglobin with different contents, establishing the relationship between reflectance and component contents by assuming a certain reasonable model, and then listing a set of equations for each pixel. In this embodiment, using the Lambert-Beer law model as a reference (actually, multiple different models can be referenced or used, as long as the assumption is reasonable), it is assumed that the contents C of hemoglobin and melanin at this pixel both conform to the reflectance R=e\hat{ }(—Ca), where a represents the absorption coefficient. It suggests that the relationship between reflectance and content is exponential function, this assumption is derived from the Lambert-Beer law, i.e. A=−log(I/I₀), where A is absorbance, I represents transmitted light, and I₀ represents incident light. Although the Lambert-Beer law describes the light transmittance of a medium while there needs to simulate the reflectance of the skin, the model can be generalized if the skin is assumed to reflect all but the absorbed part of the light. Therefore, by combining with the absorption rates of hemoglobin and melanin at two wavelengths and the hyperspectral images captured, it can perform the linear regression on each pixel (or solve the linear equation in two unknowns), and calculate the content distributions of hemoglobin and melanin furthermore, if denoted them as O(x, y) and M′(x, y) respectively, there is: −Log(R)=C_{O}O+C_{M}M, where R represents the reflectance of facial skin, O and M represent the light absorbance coefficients of hemoglobin and melanin, and C_{O} and C_{M} represent the corresponding content. As shown in the absorption coefficient curves of hemoglobin and melanin in FIG. 1, −Log(R) is uniquely determined and known for each pixel in different wavebands, as well as O and M, For each pixel, the linear equation in two unknowns of C_{O} and C_{M} can be calculated by combining the image information at two wavelengths, then, the contents or concentrations of hemoglobin and melanin at each pixel position can be obtained. Using this formula to work out I₂ and I₃, which are obtained from white balance. Due to the difference of one proportional coefficient between white balance and absolute reflectance, the resulting component content will exhibit an equal offset across the image. This is related to the exponential form of the Lambert-Beer formula, namely that if R0 is used to represent the proportional coefficient of the difference between white balance and absolute reflectance, then −Log(R/R0) is the correct left side of the equation while −Log(R/R0)=−Log(R)+Log(R0), Log(R0) here is the equal offset to the whole image. In order to obtain the correct content of hemoglobin component, using the common knowledge that hair contains only melanin but no hemoglobin, and shifting the solved O(x, y) to areas such as eyebrows to 0 to get the offset, and applying the offset to the entire image.
S404: Obtaining the melanin content difference distribution image by subtracting or dividing the I₁(x, y) by the I₂(x, y) which is processed with Gaussian blur. The melanin content difference distribution image is denoted as ΔM(x, y), namely ΔM(x, y)=I₁(x, y)−B₂(x, y) or I₁(x, y)/B₂(x, y), wherein the B₂(x, y) is the original image I₂ processed with the Gaussian blur by the Gaussian Convolution (the convolution kernel should not be too large). And the mathematical expression for the two-dimensional Gaussian kernel can be expressed as

$$G ( x , y ) = \frac{1}{2 {πσ}^{2}} {e}^{- ( {x}^{2} + {y}^{2} ) / 2 {σ}^{2}} ,$$

and the expression can be replaced by using n*n dimensional numerical matrices in practical applications. Then, it can obtain a blurred hyperspectral image B₂(x, y)=I₂(x, y)*g(x, y).
S405: Adding the calculated content distribution of melanin to the melanin content difference distribution image, obtaining the final melanin distribution image, namely M(x, y)=M′(x, y)+ΔM(x, y).
S406: Enhancing the gray-scale image of content of the hemoglobin and melanin to improve image contrast and visual display effect. Image enhancement comprises various image processing methods such as maximum and minimum normalization, contrast enhancement, and histogram equalization. Finally, obtain contrasting content images of hemoglobin and melanin components.

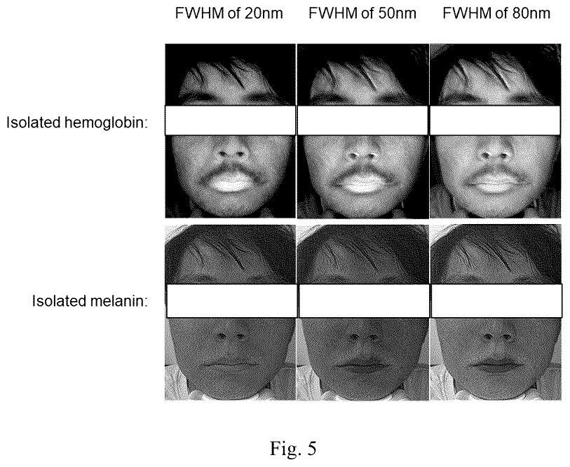

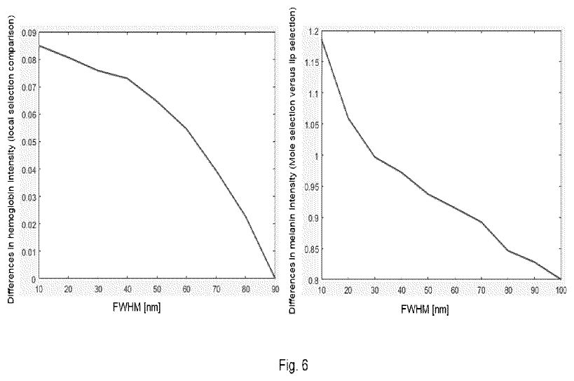

In specific embodiments, as shown in the renderings and curves in FIGS. 5 and 6, after multiple experiments and calculations by the inventors of this application, it can be seen that when the FWHM of a single waveband (which is used to measure the spectral resolution of hyperspectral imaging) is greater than 40 nm, it cannot separate effectively by using this scheme. The FWHM between 10-20 nm has the best effect. The FWHM of red, blue, and green filters are all usually higher than 80 nm in ordinary RGB cameras. It can be seen that in the narrow waveband, the melanin area of the lips appears light (for it is not a high-melanin area), while the lips will be incorrectly recognized as melanin areas when the FWHM becomes wider, due to the aliasing of spectral information collected from the image. At the same time, as the FWHM increases, the hemoglobin features separated from the image become less obvious, and the effect deteriorates. In other cases, it can also be observed that moles that originally belonged to the rich melanin area are confused as hemoglobin features in ordinary RGB camera imaging. Therefore, the method of the present invention uses a hyperspectral imaging camera with a small FWHM (such as within 50 nm) to ensure clear separation of facial components, which is rigorous in physical meaning.

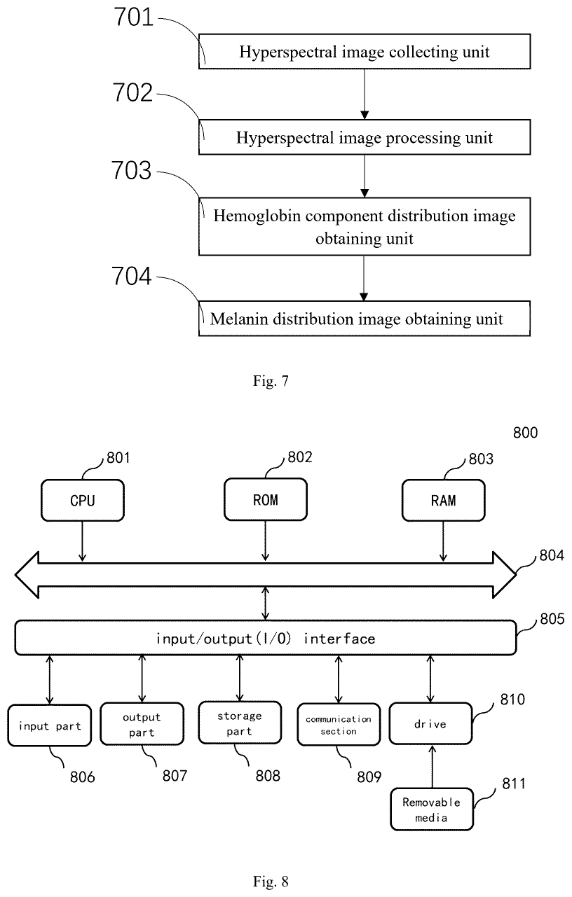

FIG. 7 illustrates a block diagram of the separation system for facial skin component image in one embodiment of the application. As shown in FIG. 7, the system comprises a hyperspectral image collecting unit 701, a hyperspectral image processing unit 702, a hemoglobin component distribution image obtaining unit 703, and a melanin distribution image obtaining unit 704. The hyperspectral image collecting unit 701 is configured for collecting facial hyperspectral image data in different preset wavebands, which wavebands have obvious relative changes of hemoglobin component and melanin. The hyperspectral image processing unit 702 is configured for performing white balance processing or absolute reflectance processing on the face hyperspectral image data in the different wavebands. The hemoglobin component distribution image obtaining unit 703 is configured for using an image processing algorithm to obtain an original hemoglobin component distribution image, constructing a skin reflection model, and obtaining content distributions of melanin and hemoglobin component by making linear regression on pixels comprising concentrations of melanin and hemoglobin component. The melanin distribution image obtaining unit 704 is configured for using an image processing algorithm to obtain a melanin content difference distribution image, and obtaining a melanin distribution image based on the content distribution of melanin component, wherein the image processing algorithm comprises image subtraction or image division.

In specific embodiments, it further comprises an image enhancement processing unit configured for performing image enhancement processing on gray-scale images of contents of hemoglobin and melanin, and the image enhancement processing comprises maximum-minimum normalization, contrast enhancement, and histogram equalization.

Referring to FIG. 8, it shows a schematic diagram of the structure of a computer system 800 suitable for implementing the electronic equipment of the embodiments of this application. The electronic device shown in FIG. 8 is only an example and should not impose any limitations on the functionality and scope of use of the embodiments of this application.

As shown in FIG. 8, the computer system 800 comprises a central processing unit (CPU) 801, which can perform various appropriate actions and processing based on programs stored in read-only memory (ROM) 802 or loaded from storage section 808 into random access memory (RAM) 803. In RAM 803, various programs and data required for the operation of the system 800 are also stored. CPU 801, ROM 802, and RAM 803 are connected to each other via bus 804. The input/output (I/O) interface 805 is also connected to bus 804.

The following parts are connected to the I/O interface 805: the input part 806 such as the keyboard, mouse, etc., and the output part 807 such as liquid crystal display (LCD), loudspeaker, etc., and the storage part 808 such as hard disk, etc., and the communication part 809 of network interface cards such as LAN card, modem, etc. The communications section 809 performs communication processing over the network such as the Internet. The drive 810 is also connected to the I/O interface 805 as required. Removable media 811, such as disk, optical disc, magnetic disc, semiconductor memory, etc., are installed on drive 810 as required, so that computer programs read from it are installed into the storage part 808 as required.

In particular, according to embodiments of the present disclosure, the process described in the reference flow chart above may be implemented as a computer software program. For example, the embodiments of the present disclosure comprise a computer program product comprising a computer program carried on a computer-readable storage medium, comprising program code for executing the method shown in the flowchart. In such embodiments, the computer program can be downloaded and installed from the network through the communication section 809, and/or installed from the removable medium 811. When the computer program is executed by the central processing unit (CPU) 801, the above-mentioned functions defined in the method of this application are executed. It should be noted that the computer readable storage medium described in this application can be a computer readable signal medium, a computer readable storage medium, or any combination of the above two. Computer readable storage media can be but not limited to, electrical, magnetic, optical, electromagnetic, infrared, or semiconductor systems, devices or devices, or any combination of the above. More specific examples of computer readable storage media may comprise but not limited to: electrical connections with one or more wires, portable computer disk, hard drive, random access memory (RAM), read-only memory (ROM), erasable programmable read-only memory (EPROM or flash), fiber optic, portable compact disk read-only memory (CD-ROM), optical storage device, magnetic storage device, or any suitable combination of the above. In this application, the computer-readable storage medium may be any tangible medium containing or storing a program, which may be used by an instruction execution system, device, or device, or in combination with it. In this application, a computer-readable signal medium may be comprised in the base waveband or as a data signal propagated as part of the carrier wave, which carries computer-readable program code. This type of transmitted data signal can take various forms, comprising but not limited to electromagnetic signal, optical signal, or any suitable combination of the above. A computer-readable signal medium can also be any computer-readable storage medium other than a computer-readable storage medium, which can send, propagate, or transmit program used by or in combination with instruction execution system, equipment, or device. The program code contained on computer-readable storage media can be transmitted with any appropriate medium, comprising but not limited to: wireless, wire, fiber optic cable, RF, etc., or any suitable combination of the above.

Computer program code for executing the operations of the present application may be written in one or more programming languages or combinations thereof, comprising object-oriented programming languages such as Java, Smalltalk, C++, and conventional procedural programming languages such as C or similar programming languages. Program code can be completely executed on the user's computer, partially executed on the user's computer, executed as an independent software package, partially executed on the user's computer, partially executed on a remote computer, or completely executed on a remote computer or server. In case involving remote computers, remote computer can connect to user computer through any type of network, comprising local area network (LAN) or wide area network (WAN), or can connect to external computer (such as using internet service providers to connect via the internet).

The flowchart and block diagram in the attached figure illustrate the possible architecture, functions, and operations of the system, methods, and computer program products according to various embodiments of the present application. In this, each box in a flowchart or block diagram can represent a module, program segment, or part of code that contains one or more executable instructions for implementing the specified logical function. It should also be noted that in some alternative implementations, the functions indicated in the box can also occur in a different order than those indicated in the accompanying drawings. For example, two consecutive boxes can actually be executed in basic parallel, and sometimes they can also be executed in opposite order, it depends on the functionality involved. It should also be noted that each box in the block diagram and/or flowchart, as well as the combination of boxes in the block diagram and/or flowchart, can be implemented using dedicated hardware based systems that perform specified functions or operations, or can be implemented using a combination of dedicated hardware and computer instructions.

The modules described in the embodiments of this application can be implemented through software or hardware.

On the other hand, the present application also provides a computer readable storage medium which may be contained in the electronic device described in the above embodiment; it can also exist alone and not be incorporated into the electronic device. The computer readable storage medium carries one or more programs, when executed by the electronic device, the programs cause the electronic device: collecting facial hyperspectral image data in different preset wavebands, which wavebands have obvious relative changes of hemoglobin component and melanin; performing white balance processing or absolute reflectance processing on the face hyperspectral image data in the different wavebands; using an image processing algorithm to obtain an original hemoglobin component distribution image, constructing a skin reflection model, and obtaining content distributions of melanin and hemoglobin component by making linear regression on pixels comprising concentrations of melanin and hemoglobin component; using an image processing algorithm to obtain a melanin content difference distribution image, and obtaining a melanin distribution image based on the content distribution of melanin component, wherein the image processing algorithm comprises image subtraction or image division.

The above description is only a preferred embodiment of this application and an explanation of the technical principles used. Technicians in this field should understand that the scope of the invention referred to in this application is not limited to technical solutions formed by specific combinations of the aforementioned technical features, and should also cover other technical solutions formed by arbitrary combinations of the aforementioned technical features or their equivalent features without departing from the aforementioned invention concept. For example, it can be a technical solution formed by replacing the above features with (but not limited to) technical features with similar functions disclosed in this application.

1. A separation method for image with facial skin component, comprising the following steps of:
S1: collecting facial hyperspectral image data in different preset wavebands, which wavebands have obvious relative changes of hemoglobin component and melanin;
S2: performing white balance processing or absolute reflectance processing on the face hyperspectral image data in the different wavebands;
S3: using an image processing algorithm to obtain an original hemoglobin component distribution image, constructing a skin reflection model, and obtaining content distributions of melanin and hemoglobin component by making linear regression on pixels comprising concentrations of melanin and hemoglobin component; and
S4: using an image processing algorithm to obtain a melanin content difference distribution image, and obtaining a melanin distribution image based on the content distribution of melanin component, wherein the image processing algorithm comprises image subtraction or image division.

2. The separation method for image with facial skin component according to claim 1, wherein the facial hyperspectral image data is taken by a hyperspectral imaging camera with a Full Width at Half Maximum (FWHM) less than 50 nm.

3. The separation method for image with facial skin component according to claim 1, wherein the facial hyperspectral image data of three different preset wavebands is collected, and the white balance processing in step S2 comprises: obtaining the white balance by using the Gray World Algorithm, and obtaining three frames of facial hyperspectral images I1(x, y), I2(x, y), and I3(x, y) after the white balance.

4. The separation method for image with facial skin component according to claim 1, wherein the facial hyperspectral image data of three preset different wavebands is collected, and the absolute reflectance processing in step S2 comprises: obtaining hyperspectral data of at original facial position of a reference white board in corresponding waveband, dividing the facial hyperspectral images by the hyperspectral data of the reference white board, and obtaining three frames of facial hyperspectral images I1(x, y), I2(x, y), and I3(x, y) after the absolute reflectance processing.

5. The separation method for image with facial skin component according to claim 3, wherein the three preset different wavebands comprise 530-560 nm, 575-585 nm and 600-630 nm, and the three frames of facial hyperspectral images in the corresponding three wavebands are obtained.

6. The separation method for image with facial skin component according to claim 3, wherein the using an image processing algorithm to obtain an original hemoglobin component distribution image, comprises: subtracting the facial hyperspectral image I2(x, y) by I3(x, y), or dividing I2(x, y) by I3(x, y) to obtain the original hemoglobin component distribution image O(x, y).

7. The separation method for image with facial skin component according to claim 1, wherein the constructing a skin reflection model comprises: based on the Lambert-Beer law, linear regression is performed on each of the pixels by combining light absorbance values of hemoglobin and melanin in two wavebands and the hyperspectral image data captured: −Log(R)=COO+CMM, image information in two wavebands is used to obtain linear equation in two unknowns of CO and CM, and the concentration or content of hemoglobin and melanin at each pixel position is obtained, where R represents the reflectance of facial skin, O and M represent the light absorbance coefficients of hemoglobin and melanin respectively, CO and CM represent the corresponding concentration or content of hemoglobin and melanin.

8. The separation method for image with facial skin component according to claim 3, wherein obtaining the melanin content difference distribution image in the step S4 comprises: obtaining the melanin content difference distribution image ΔM(x, y) by subtracting the facial hyperspectral image I1(x, y) by the I2(x, y) which is processed with Gaussian blur, or by dividing the I1(x, y) by the I2(x, y) which is processed with Gaussian blur.

9. The separation method for image with facial skin component according to claim 8, wherein the obtaining the melanin distribution image comprises: obtaining the melanin distribution image M(x, y) based on the image processing algorithm, M(x, y)=I3(x, y)+ΔM(x, y), or obtaining the melanin distribution image M(x, y) based on the skin reflection model, M(x, y)=M′(x, y)+ΔM(x, y), where M′(x, y) is the content distribution of melanin component obtained by the linear regression.

10. The separation method for image with facial skin component according to claim 1, wherein further comprises image enhancement processing on gray-scale images of contents of hemoglobin and melanin, and the image enhancement processing comprises maximum-minimum normalization, contrast enhancement, and histogram equalization.

11. A separation system for image separation with facial skin component, comprising:
hyperspectral image collecting unit: configured for collecting facial hyperspectral image data in different preset wavebands, which wavebands have obvious relative changes of hemoglobin component and melanin;
hyperspectral image processing unit: configured for performing white balance processing or absolute reflectance processing on the face hyperspectral image data in the different wavebands;
hemoglobin component distribution image obtaining unit: configured for using an image processing algorithm to obtain an original hemoglobin component distribution image, constructing a skin reflection model, and obtaining content distributions of melanin and hemoglobin component by making linear regression on pixels comprising concentrations of melanin and hemoglobin component; and
melanin distribution image obtaining unit: configured for using an image processing algorithm to obtain a melanin content difference distribution image, and obtaining a melanin distribution image based on the content distribution of melanin component, wherein the image processing algorithm comprises image subtraction or image division.

12. The separation system for image separation with facial skin component according to claim 11, further comprising an image enhancement processing unit configured for performing image enhancement processing on gray-scale images of contents of hemoglobin and melanin, and the image enhancement processing comprises maximum-minimum normalization, contrast enhancement, and histogram equalization.

13. (canceled)

14. A non-transitory computer-readable medium for storing executable instructions, which cause a method to be performed, the method comprises the following steps of:
S1: collecting facial hyperspectral image data in different preset wavebands, which wavebands have obvious relative changes of hemoglobin component and melanin;
S2: performing white balance processing or absolute reflectance processing on the face hyperspectral image data in the different wavebands;
S3: using an image processing algorithm to obtain an original hemoglobin component distribution image, constructing a skin reflection model, and obtaining content distributions of melanin and hemoglobin component by making linear regression on pixels comprising concentrations of melanin and hemoglobin component; and
S4: using an image processing algorithm to obtain a melanin content difference distribution image, and obtaining a melanin distribution image based on the content distribution of melanin component, wherein the image processing algorithm comprises image subtraction or image division.

15. The separation method for image with facial skin component according to claim 4, wherein the three preset different wavebands comprise 530-560 nm, 575-585 nm and 600-630 nm, and the three frames of facial hyperspectral images in the corresponding three wavebands are obtained.

16. The separation method for image with facial skin component according to claim 4, wherein the using an image processing algorithm to obtain an original hemoglobin component distribution image, comprises: subtracting the facial hyperspectral image I2(x, y) by I3(x, y), or dividing I2(x, y) by I3(x, y) to obtain the original hemoglobin component distribution image O(x, y).

17. The separation method for image with facial skin component according to claim 4, wherein obtaining the melanin content difference distribution image in the step S4 comprises: obtaining the melanin content difference distribution image ΔM(x, y) by subtracting the facial hyperspectral image I1(x, y) by the I2(x, y) which is processed with Gaussian blur, or by dividing the I1(x, y) by the I2(x, y) which is processed with Gaussian blur.
